(19) United States
(12) Patent Application Publication
(10) Pub. No.: US 2007/0219383 A1
(43) Pub. Date: Sep. 20, 2007

(54) Method for Reducing Alkyne Compuonds

(76) Inventors: Johannes Grimmer, Ludwigshafen (DE); Thomas Muller, Dirmstein (DE)

Correspondence Address: CONNOLLY BOVE LODGE & HUTZ, LLP, P O BOX 2207, WILMINGTON, DE, 19899, US

(21) Appl. No.: 11/662,440

(22) PCT Filed: Sep. 8, 2005
(86) PCT No.: PCT/EP05/09655

§ 371 Date: Mar. 9, 2007

(30) Foreign Application Priority Data
Sep. 10, 2004 (DE) 102004044262.2

(51) Int. Cl. C07D 317/64; C01C 1/00; C07C 403/08; C07C 45/00

(52) U.S. Cl. 549/430; 423/356; 568/338; 585/360

(57) ABSTRACT

The present invention relates to a method for preparing cyclohexene derivatives of the general formulae I or II 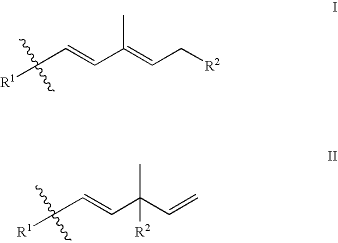
by reducing alkyne compounds of the general formulae III or IV 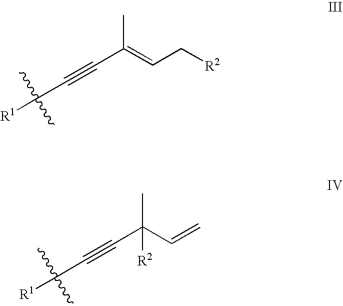
with a mixture of zinc and at least one ammonium salt of the formula V as reducing agent, 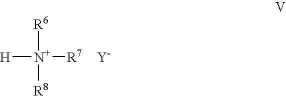
in which the substituents R¹ to R⁸ have independently of one another the meaning specified in the description, wherein the reducing agent comprises from 0.3 to 0.49 mol of at least one ammonium salt of the formula V per mol of zinc.

The present invention relates to a novel method for reducing alkyne compounds, in particular the invention relates to a method for preparing cyclohexene derivatives which are suitable as intermediates for preparing carotenoids.

A large number of the industrial carotenoid syntheses described in the literature, including the preparation of astaxanthin, proceeds via cyclohexene intermediates which, besides one or more C═C double bonds, also comprise a C5C triple bond. To form a conjugated double-bond system it is necessary for this triple bond to be partially reduced in a separate step of the method.

This can take place in the context of the astaxanthin synthesis as those described in DE-A-43 22 277 in the case of the alkynediol IVa with zinc/acetic acid in methylene chloride.

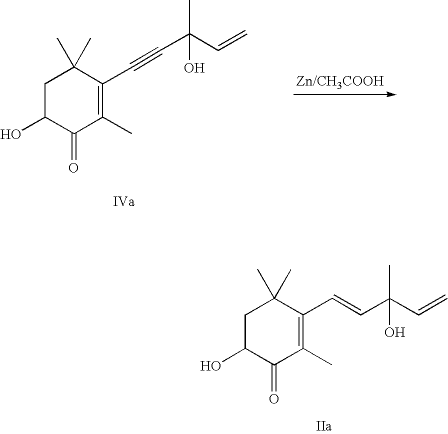

EP-A-0 005 748 relates to a further method for preparing astaxanthin, in which the partial reduction of the alkynediol of the formula IIIa is likewise carried out with zinc/acetic acid in methylene chloride.

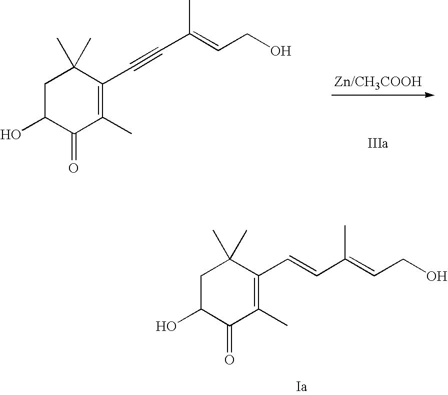

One disadvantage of the described zinc/acetic acid reduction is the inadequate selectivity of the method. Unwanted by-products such as, for example, the formation of spiro compounds, which cannot be converted in the subsequent course of the synthesis into the desired following products can only lead to significant losses of yield.

Further reduction methods are described inter alia in J. Amer. Oil Chem. Soc. 49 (1972) 72, in which the reduction of triple bonds to cis double bonds takes place in long-chain, conjugated fatty acids with zinc in boiling protic solvents.

The drastic reduction conditions mentioned herein are unsuitable for thermally unstable compounds.

Helv. Chim. Acta 58 (1975) 1016 describes the reduction of conjugated alkynes in protic solvents. The reducing agent used by the authors is zinc dust which has been activated by adding potassium cyanide.

The abovementioned methods on the one hand afford only moderate yields and, on the other hand, activation with potassium cyanide leads to a considerable health risk.

The publication in the Journal fur praktische Chemie 336 (1994) 714-715 includes a method for the (Z)-selective reduction of conjugated triple bonds with a combination of Zn (Cu/Ag) in polar protic solvents such as, for example, methanol/water.

The disadvantage of this method is that the preparation of the reagent is vry complicated and, moreover, the reagent must always be prepared freshly.

EP 1 197 483 A2 describes a method for the catalytic reduction of alkyne compounds which comprises using as reducing agent a mixture of zinc and at least one compound selected from the group consisting of ammonium salts, copper salts, alkali metal and alkaline earth metal salts.

The object of the present invention was therefore to provide a method for the partial reduction of alkyne compounds with which the abovementioned disadvantages of the prior art are avoided.

This object has been achieved by a method for preparing cyclohexene derivatives of the general formulae I or II, 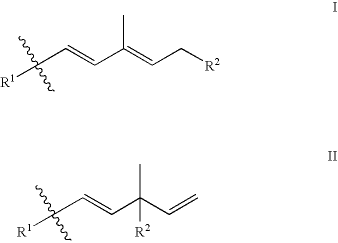
in which the substituents R¹ and R² have independently of one another the following meaning: 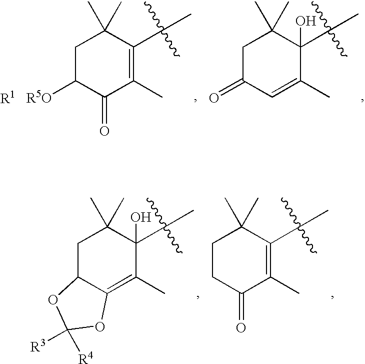
R² OH or a protective group which can be converted into a hydroxy group by hydrolysis,
R³and R⁴
hydrogen, C₁-C₄-alkyl;
R⁵ hydrogen, C₁-C₄-acyl;
by reducing alkyne compounds of the general formulae III or IV, 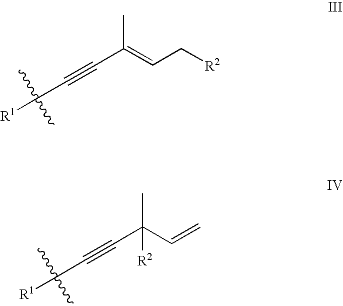
   in which the substituents R¹ and R² have the abovementioned meaning, with a mixture of zinc and at least one ammonium salt of the formula V 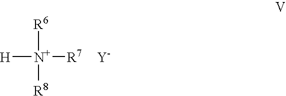
   in which the substituents have independently of one another the following meaning:
R⁶ to R⁸ hydrogen, C₁-C₆-alkyl, aryl;
Y⁻ anion of an organic or inorganic acid,
wherein the reducing agent comprises from 0.3 to 0.49 mol of at least one ammonium salt of the formula V per mol of zinc.

Alkyl radicals which may be mentioned for R³ and R⁴ are linear or branched C₁-C₄-alkyl chains, e.g. methyl, ethyl, n-propyl, 1-methylethyl, n-butyl, 1-methylpropyl, 2-methylpropyl and 1,1-dimethylethyl. Methyl and ethyl are preferred alkyl radicals.

The radicals R³ and R⁴ may also form together with the carbon atom to which they are bonded a cycloheptyl or cyclohexyl ring.

Substituents which may be mentioned for R⁵ are linear or branched C₁-C₄-acyl chains, e.g. formyl, acetyl, propionyl, isopropionyl. The preferred acyl radical is acetyl.

Functional groups suitable for a protective group for R² which can be converted into a hydroxy group by hydrolysis are those which can be converted relatively easily into the hydroxy group. Examples which may be mentioned are ether groups such as 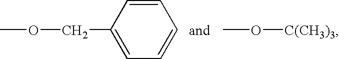
silyl ether groups such as —O—Si(CH₃)₃, —O—Si(CH₂CH₃)₃, —O—Si(isopropyl)₃, —O—Si(CH₃)₂(tert-butyl) and —O—Si(CH₃)₂(n-hexyl) or substituted methyl ether groups such as the a-alkoxyalkyl ether groups of the formulae 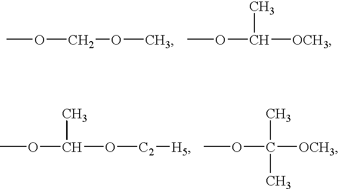
and suitable pyranyl ether groups such as the tetrahydropyranyloxy group and the 4-methyl-5,6-dihydro-2H-pyranyloxy group.

It is particularly advantageous to use for R² the tetrahydropyranyloxy group 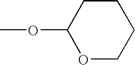
or the α-ethoxyethoxy group of the formula

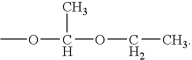

Conditions for eliminating the abovementioned protective groups are to be found inter alia in T. Greene “Protective Groups in Organic Chemistry”, John Wiley & Sons, 1981, Chapter 2.

Alkyl radicals which may be mentioned for R⁶ to R⁸ are linear or branched C₁-C₆-alkyl chains, e.g. methyl, ethyl, n-propyl, 1-methylethyl, n-butyl, 1-methylpropyl-, 2-methylpropyl, 1,1-dimethylethyl, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl. Preferred alkyl radicals are methyl, ethyl, n-propyl and 1-methylethyl.

Hydrogen is to be mentioned as particularly preferred radical for R⁶ to R⁸.

Aryl means aromatic rings or ring systems having 6 to 18 carbon atoms in the ring system, for example phenyl or naphthyl, which may optionally be substituted by one or more radicals such as halogen, e.g. fluorine, chlorine or bromine, amino, C₁-C₄-alkyl-amino, C₁-C₄-dialkylamino, hydroxy, C₁-C₄-alkyl, C₁-C₄-alkoxy or other radicals. Optionally substituted phenyl, methoxyphenyl and naphthyl are preferred.

Y⁻ is generally an anion of an organic or inorganic acid.

Organic acids mean inter alia aliphatic and aromatic carboxylic acids, for example benzoic acid or C₁-C₁₂alkanoic acids, preferably C₁-C₆-alkanoic acids such as formic acid, acetic acid, propionic acid, butyric acid, and caproic acid, particularly preferably acetic acid or dicarboxylic acids such as oxalic acid, malonic acid and succinic acid.

It is also possible for Y⁻ to be anions of organic sulfonic acids such as methanesulfonate or para-toluenesulfonate.

Examples of inorganic acids are inter alia hydrochloric acid, hydrobromic acid, carbonic acid, sulfuric acid, sulfurous acid, nitric acid, nitrous acid and phosphoric acid.

A particularly preferred variant of the method comprises using as reducing agent a mixture of zinc and at least one ammonium salt of the formula V selected from the group consisting of ammonium chloride, ammonium carbonate, ammonium bicarbonate, ammonium sulfate and ammonium acetate. The substituents R⁶ to R⁸ are in this case jointly hydrogen.

Ammonium chloride may be mentioned as very particularly preferred ammonium salt.

The method of the invention is particularly suitable for preparing the cyclohexene compounds of the formulae Ia and IIa.

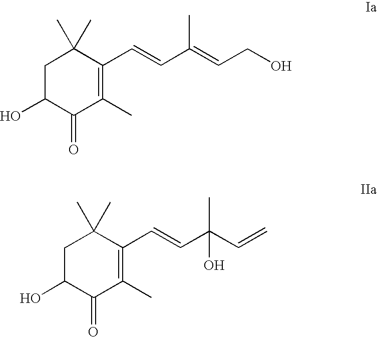

The procedure for carrying out the method is generally such that an aqueous solution of at least one ammonium salt of the formula V is metered into the alkyne compounds of the formulae III or IV, and then the zinc is added to this mixture, or a suspension of zinc in the aqueous solution of at least one ammonium salt of the formula V is metered into the abovementioned alkyne compounds.

However, an inverse procedure is also possible, where the zinc is suspended in an aqueous solution of at least one ammonium salt of the formula V, and the alkyne compounds III or IV are added to this suspension.

It has further emerged that the reduction according to the invention takes place particularly advantageously in the presence of water.

The amount of water is chosen so that the compound B is in dissolved or partly dissolved form. Ordinarily, from 15 to 500 ml of water, preferably 20 to 400 ml, particularly preferably 30 to 250 ml, of water are used per mole of zinc employed.

It has been realized that addition of an inert solvent is a further advantage for the course of the reduction.

In general all solvents inert for the compounds I to IV are suitable as inert solvent in the method of the invention. It is preferable to use chlorinated hydrocarbons such as, for example, dichloromethane, perchloroethylene or chloroform or an ethereal solvent such as dialkyl ethers, tetrahydrofuran or dioxane, especially the water-immiscible methyl tert-butyl ether. Further solvents which are also suitable are aromatic hydrocarbons, especially toluene, and C₁-C₃ alcohols such as methanol, ethanol or propanol.

It is preferred to use a 10 to 50% by weight solution of the alkynediol in one of the abovementioned solvents, particularly preferably a 15 to 30% by weight solution of the alkynediol in methylene chloride.

It is also possible to employ acetic acid as cosolvent in addition to the abovementioned solvents.

The zinc employed is employed in an amount of about 0.5 to 5, preferably 0.7 to 3, particularly preferably 1 to 2, very particularly preferably 1.1 to 1.5, gram atoms per mole of the alkynediol to be reduced. Metering in of the zinc in one or more portions is also possible.

From 0.3 to 0.49 mole, preferably 0.35 to 0.45 mole, particularly preferably 0.4 mole, of at least one ammonium salt of the formula V is employed per mole of zinc.

The reduction can be carried out at temperatures between 0° C. and the boiling point of the appropriate solvent. Preferred reaction temperatures are in the range from 10 to 80° C., particularly preferably in the range 35-45° C.

The advantage of the method of the invention over the preparation processes mentioned in the prior art is inter alia that the selectivity for the desired product is higher and less salt is produced, and thus the preparation process is more economic.

The subject matter of the present invention is to be explained in more detail by means of the following examples.

### EXAMPLE 1

49.6 g (0.2 mole) of 6-hydroxy-3-(3-hydroxy-3-methyl-4-penten-1-ynyl)-2,4,4-trimethyl-2-cyclohexen-1-one of the formula IVa with a purity of 92% were dissolved in 100 g of methylene chloride and mixed with a solution of 5.14 g (0.096 mole) of ammonium chloride in 43.2 ml of water. The mixture was heated to 38° C., and 4 portions each of 3.9 g (0.24 mole) of zinc powder were added over the course of 4 hours. After a reaction time (including introduction of zinc) of 5 hours, a sample was taken and the selectivity of the reaction for the alkenediol of the formula IIa was determined by gas chromatographic analysis to be 85.4%.

### EXAMPLE 2

49.6 g (0.2 mole) of 6-hydroxy-3-(3-hydroxy-3-methyl-4-penten-1-ynyl)-2,4,4-trimethyl-2-cyclohexen-1-one of the formula IVa with a purity of 92% were dissolved in 100 g of methylene chloride and mixed with a solution of 3.85 g (0.072 mole) of ammonium chloride in 43.2 ml of water. The mixture was heated to 38° C., and 4 portions each of 3.9 g (0.24 mole) of zinc powder were added over the course of 4 hours. After a reaction time (including introduction of zinc) of 5 hours, a sample was taken and the selectivity of the reaction for the alkenediol of the formula Ia was determined by gas chromatographic analysis to be 80%.

### EXAMPLE 3

49.6 g (0.2 mole) of 6-hydroxy-3-(3-hydroxy-3-methyl-4-penten-1-ynyl)-2,4,4-trimethyl-2-cyclohexen-1-one of the formula IVa with a purity of 92% were dissolved in 100 g of methylene chloride and mixed with a solution of 6.42 g (0.12 mole) of ammonium chloride in 43.2 ml of water. The mixture was heated to 38° C., and 4 portions each of 3.9 g (0.24 mole) of zinc powder were added over the course of 4 hours. After a reaction time (including introduction of zinc) of 5 hours, a sample was taken and the selectivity of the reaction for the alkenediol of the formula IIa was determined by gas chromatographic analysis to be 80.63%.

1-5. (canceled)

6. A method for preparing cyclohexene derivatives of the general formulae I or II, 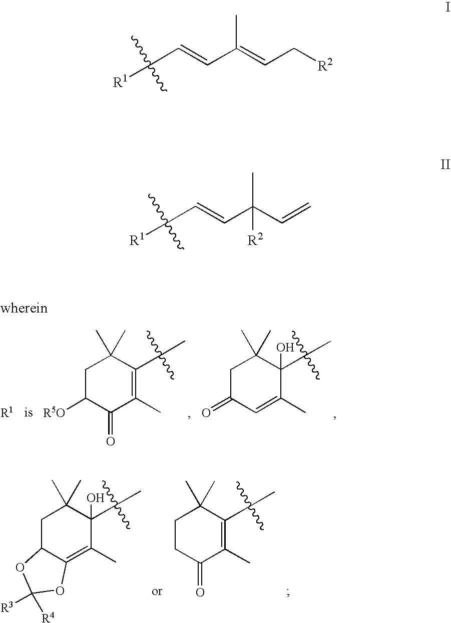
R² is OH or a protective group which can be converted into a hydroxy group by hydrolysis,
R³ and R⁴ are, independently of one another, H or C₁-C₄-alkyl; and
R⁵ is H or C₁-C₄-acyl;
comprising the step of reducing alkyne compounds of the general formulae III or IV, 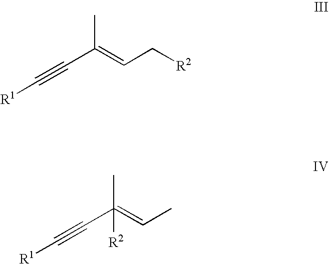
wherein R¹ and R² are as defined above, with a mixture of zinc and at least one ammonium salt of the formula V 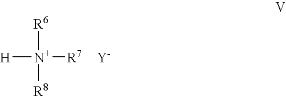
wherein
R⁶, R⁷, and R⁸ are, independently of one another, H, C₁-C₆-alkyl, or aryl; and
Y⁻ is an anion of an organic or inorganic acid;
wherein the reducing agent comprises from 0.3 to 0.49 mol of at least one ammonium salt of the formula V per mol of zinc.

7. The method according to claim 6, wherein said at least one ammonium salt of the formula V is selected from the group consisting of ammonium chloride, ammonium carbonate, ammonium bicarbonate, ammonium sulfate, and ammonium acetate.

8. The method according to claim 6, wherein the reduction is carried out in the presence of water.

9. The method according to claim 6, wherein the reduction is carried out in an organic solvent which is inert to the compounds of general formulae I, II, III, and IV.

10. The method according to claim 6, wherein cyclohexene derivatives of the formulae Ia and IIa 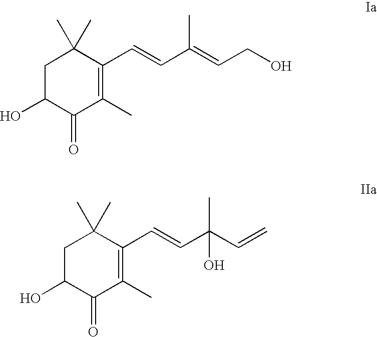
are prepared.
